(19) United States
(12) Patent Application Publication
(10) Pub. No.: US 2019/0365544 A1
(43) Pub. Date: Dec. 5, 2019

(54) EXPANDABLE SPINAL IMPLANTS AND INSTRUMENTS AND METHODS FOR IMPLANT DELIVERY AND DEPLOYMENT

(71) Applicant: Javier GARCIA-BENGOCHEA, Jacksonville, FL (US)

(72) Inventor: Javier GARCIA-BENGOCHEA, Jacksonville, FL (US)

(21) Appl. No.: 16/478,685

(22) PCT Filed: Jan. 19, 2018
(86) PCT No.: PCT/US18/14527

§ 371 Date: Jul. 17, 2019

Related U.S. Application Data
(60) Provisional application No. 62/448,267, filed on Jan. 19, 2017.

(51) Int. Cl. A61F 2/44; A61F 2/46

(52) U.S. Cl. CPC A61F 2/4455; A61B 17/7059; A61F 2/4611

(57) ABSTRACT

Novel implants, instruments and surgical techniques are provided for manipulating a spinal disc space in order to one or more of: optimize disc space preparation; optimize the positioning of implants within the disc space; achieve and maintain distraction during interbody placement; and provide sagittal balance during fusion.

### RELATED APPLICATIONS

This application is a United States National Stage Application filed under 35 U.S.C. 371 and claims priority to PCT Patent Application No. PCT/US2018/014527 filed Jan. 19, 2018, which application claims the benefit under 35 U.S.C. § 119(e) to U.S. Provisional Application Ser. No. 62/448,267 filed Jan. 19, 2017, the entirety of which applications are incorporated herein by reference.

### FIELD

The present application describes various exemplary instruments, implants and surgical techniques for achieving access to and placement of implants at a site within the body, particularly the spine. More particularly, the present application describes instruments useful for accessing the spine for one or more purposes of preparation of the disc space, placement, in situ assembly, and manipulation of intervertebral implants to supplement or replace natural spinal discs, particularly in the lumbar spine. The instruments are particularly useful for distraction of the disc space and disruption of the annulus, particularly the anterior annulus. The implants and instruments for manipulation thereof enable controlled positioning, assembly and deployment, particularly but not limited to, by a transforaminal mode of access, of implants within the intervertebral space so as to enable anterior oriented placement of an implant within the disc space of the spine to achieve enhanced lordosis and sagittal balance.

### DESCRIPTION OF THE RELATED ART

Surgical access to the spine is achieved by a variety of different access routes, some or all of which involve complications and drawbacks depending on the condition of the patient, the extent of the disease or dysfunction, and the skill of the surgeon, among other factors. Access via anterior and posterior routes have certain advantages, but they are not necessarily minimally invasive, and involve some meaningful disadvantages, including the need for multiple surgeons (in the case of anterior access) and the need for creation of multiple openings into the disc space and related dura and soft tissue disruption (in the case of posterior access), among others.

Surgeons have evolved other modes of access, particularly for the lumbar spine, for example, through foramen of the spine (referred to as transforaminal lumbar interbody fusion, or TLIF). This mode of access involves unilateral entry into the disc space from a generally posterior approach that is affected by removing part or all of a single facet joint on one side of the spine to form a channel into the intervertebral space. This approach does not require dura retraction, it is not bilateral, it avoids muscle and soft tissue damage, and can be completed by a single surgeon.

Despite the described advantages to TLIF, there are drawbacks. For example, the channel through the resected facet is small, and presents only limited space for insertion of instruments and implants. This translates to what can be significant difficulty implant manipulation once an interbody device is placed in the disc space. And, because the implants are relatively small, and thus cover a relatively small fraction of the surfaces of the endplates, there is a fairly high risk of failure as a result of collapse of the endplates around the implant. Thus, it is desirable to have an enhanced means for preparation of the disc space. And in some instances, it is desirable to provide implants and instruments to enable the placement and in situ assembly of implant. Such a system would permit desirable distribution of an implant or implant assembly across the disc space and achieve better support between the vertebrae and resultant more stable fusion, while taking advantage of the minimally invasive aspects of TLIF and related spinal access approaches.

### SUMMARY

In accordance with the disclosure, novel implants, as well as instruments and a surgical technique are provided for manipulating the disc space in order to optimize the positioning of implants within the disc space to enable optimized disc space preparation, achieve and maintain distraction during interbody placement, and provide sagittal balance during fusion. As an aspect of a fusion procedure, disrupting the anterior annulus—known as ACR—Anterior Column Release—is known to be an essential component to restoring lumbar lordosis, but presents particular challenges especially in the context of minimally invasive surgical access, such as via a TLIF technique. Inadequate lordosis is attributable to a variety of factors, including the adequate release of the anterior annulus, placement and area of implant, among others. According the disclosed inventive embodiments, optimized release of the anterior annulus can be achieved even within the constraints of minimally invasive procedures, and the inventive implants enhance the achievement of anatomically correct lordosis/kyphosis within the spine.

In some embodiments, provided herein is a modular spinal implant that includes a transverse component having a top surface, a bottom surface, and an emplacement and a sagittal component that has a generally wedge-shaped conformation defined by elongate sides and a wide base that tapers toward a tip portion, and a coupling surface at the base. The coupling surface of the sagittal component and the emplacement of the transverse component are arranged and disposed to engaged to form an attached configuration between the transverse component and the sagittal component, with the tip portion of the sagittal component extending from the bottom surface of the transverse component.

In some embodiments, the transverse component has a generally convex top surface. And in some particular embodiments, the transverse component is arcuate.

In some embodiments, the sagittal component includes a wedge member and a locking member, the locking member attaching to the wedge member in a locked configuration to retain the transverse component and the sagittal component in the attached configuration.

In some embodiments, the one of the wedge member and the locking member includes a gear rack and the other of the wedge member and the locking member includes a ratchet. In yet other embodiments, the wedge member includes an internal threaded surface and the locking member includes an external threaded surface.

In some embodiments, the wedge member includes a slot that opens at the tip portion and extends to the coupling surface disposed, and a first prong and a second prong on either side of the slot and extending away from the coupling surface. In some such embodiments, the slot that extends to the coupling surface is generally U shaped. In some embodiments, the locking member is arranged and disposed to occupy and substantially fill the slot in the locked configuration.

In some embodiments, the transverse component includes a first aperture and a second aperture adjacent to the emplacement, the first aperture being arranged and disposed to receive the first prong and the second aperture being arranged and disposed to receive the second prong in the attached configuration such that the coupling surface of the sagittal component contacts the emplacement of the transverse component. In some embodiments, the first aperture and the second aperture extend from the emplacement to a lateral surface of the transverse component. In some embodiments, the generally convex top surface of the transverse component includes a recess arranged and disposed to engage the base of the slot in the attached configuration.

In some embodiments, the sagittal component and the transverse component are arranged and disposed such that in the attached configuration a center line of the sagittal component extending from the bottom surface of the transverse component to the tip portion of the sagittal component is oblique relative to the bottom surface.

In some embodiments, the coupling surface of the sagittal component is on the base and includes an attachment feature that is complimentary with an attachment feature on the bottom surface of the transverse

In some embodiments, transverse component has a length dimension, a width dimension and a depth dimension, and wherein the length dimension is greater than the width dimension which is greater than the depth dimension. In some embodiments, the transverse component includes a first aperture and a second aperture adjacent to the emplacement to define two planar members, wherein the apertures and the emplacement are either centered or off center along the length dimension. In some embodiments, the transverse component has a generally arcuate shape.

In an exemplary embodiment, the modular implant includes a transverse component having a length dimension, a width dimension and a depth dimension, wherein the length dimension is greater than the width dimension, which is greater than the depth dimension, and having two planar members defined by apertures adjacent to the emplacement. The modular implant further includes a sagittal component that includes a wedge member having a slot that opens at the tip portion and extends to the coupling surface to define a first prong and a second prong on either side of the slot and extending away from the coupling surface, wherein the slot has a width dimension between the prongs that is larger than the depth dimension of the transverse component and is smaller than the width dimension of the transverse component. According to such embodiment, the transverse component is adapted for insertion into the slot of the sagittal component lengthwise with its length dimension parallel with the prongs, and then rotated by about 90 degrees so that its length dimension is perpendicular to the prongs and its width dimension is parallel with the prongs, and then rotated again by about 90 degrees so that its depth dimension is parallel with the prongs such that the transverse component is oriented generally orthogonally with respect to the sagittal component.

In yet another embodiment, provided is a method for surgically modifying a spine with a modular spinal implant. The method includes steps including inserting a transverse component into a distracted disc space of a spinal column, the transverse component having a generally convex top surface, a bottom surface, and an emplacement. The method further includes inserting a sagittal component into the distracted disc space, the sagittal component having a generally wedge-shaped conformation defined by elongate sides and a wide base that tapers toward a tip portion, and a coupling surface. The method further includes attaching the sagittal component to the transverse component in the distracted disc space to form the modular spinal implant in an attached configuration with the coupling surface of the sagittal component engaged to the emplacement of the transverse component, and with the sagittal component extending from the bottom surface of the transverse component with the tip portion of the sagittal component distal from the bottom surface of the transverse component.

In some embodiments of the method, the sagittal component includes wedge member and a locking member and attaching the sagittal component to the transverse component includes attaching the locking member to the wedge member to form a locked configuration retaining the transverse component and the sagittal component in the attached configuration.

In some embodiments of the method, the wedge member is inserted into the distracted disc space prior to the transverse component being inserted into the distracted disc space.

In some embodiments of the method, the locking member is inserted into the distracted disc space following the transverse component being inserted into the distracted disc space.

In an exemplary embodiment, the method includes the features wherein the transverse component includes a first aperture and a second aperture adjacent to the emplacement and the wedge member includes a slot that opens at the tip portion and extends to a base of the slot, and a first prong and a second prong on either side of the slot, the method further including the step of attaching the sagittal component to the transverse component includes inserting the first prong into the first aperture and the second prong into the second aperture such that the coupling surface of the sagittal component contacts the emplacement of the transverse component.

In an exemplary embodiment of the method the first aperture and the second aperture extend from the emplacement to a lateral surface of the transverse component, and the method further includes the step of inserting the first prong into the first aperture and the second prong into the second aperture and further includes rotating the transverse component relative to the wedge member and then inserting the locking member into the slot. In some embodiments, inserting the locking member into the slot occupies and substantially fills the slot.

In some embodiments according to the method, inserting the first prong into the first aperture and the second prong into the second aperture includes engaging the base of the slot with a recess disposed in the generally convex top surface of the transverse component.

In some embodiments according to the method, either: one of the wedge member and the locking member includes a gear rack and the other of the wedge member and the locking member includes a ratchet, and attaching the locking member to the wedge member includes engaging the ratchet and the gear rack; or the wedge member includes an internal threaded surface and the locking member includes an external threaded surface, and attaching the locking member to the wedge member includes engaging the external threaded surface with the internal threaded surface.

In yet another embodiment, provided is a method for surgically preparing a spinal disc space, comprising steps that include: (i) directing at least a first elongate distractor blade having a proximal end and a distal end comprising a flared paddle into a spinal disc space and between adjacent vertebral endplates, the flared paddle of the distractor blade being parallel to the endplates; (ii) advancing the flared paddle into contact with an anterior annulus of the spinal disc space; (iii) actuating the distractor blade by rotation of its proximal end by about 90 degrees whereby the flared paddle is oriented rotated to be approximately perpendicular to the vertebral endplates; (iv) optionally introducing at least a second elongate distractor blade adjacent to the first distractor blade, and repeating each of steps (i), (ii) and (iii), the at least second distractor blade having a flared paddle that is one of dimensionally larger, equivalent to or smaller than the flared paddle of the first distractor; (v) optionally repeating step (vi) with one or more additional elongate distractor blades, each one or more additional elongate distractor blades having a flared paddle that one of dimensionally larger, equivalent to or smaller than the flared paddle of a previously inserted elongate distractor blade; (vi) withdrawing each of the one or more of the elongate distractor blades; and (vii) optionally, repeating the steps (i)-(v), one or more times.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows 6 alternate views from top to bottom, of a paddle distractor instrument according to the disclosure;

FIG. 2 shows a schematic of a set of retractors in the context of an axial (AP) radiographic image of a spine;

FIG. 3 shows a schematic of a retractor in the context of a sagittal (lateral) radiographic image of a spine;

FIG. 4 shows a sagittal (lateral) radiographic image of a spine showing a retractor inserted into the depicted disc space adjacent to the anterior annulus/anterior edges of the vertebral discs;

FIG. 5 A shows a side view of an implant according to the disclosure;

FIG. 5 B shows a first end view of an implant according to the disclosure;

FIG. 5 C shows a top perspective of an implant according to the disclosure;

FIG. 5 D shows a bottom perspective view of an assembled implant that includes each of first, second and third components fully assembled, according to the disclosure;

FIG. 6 shows in the upper panel, side, first end, bottom, second end, and cross-sectional views of a first component of the implant embodiment depicted in FIG. 5 A-FIG. 5D, and in the lower panel, first side, second side cross sectional, tope end, second side, and bottom end views of a third component of the implant embodiment depicted in FIG. 5 A-FIG. 5D;

FIG. 7 A shows a bottom view of a second component of the implant embodiment depicted in FIG. 5 A-FIG. 5D;

FIG. 7 B shows a top perspective view of a second component of the implant embodiment depicted in FIG. 5 A-FIG. 5D;

FIG. 7 C shows a side view of a second component of the implant embodiment depicted in FIG. 5 A-FIG. 5D;

FIG. 7 D shows a first end cross sectional view of the implant embodiment depicted in FIG. 5 A-FIG. 5D;

FIG. 7 E shows a second end cross sectional view of the implant embodiment depicted in FIG. 5 A-FIG. 5D;

FIG. 7 F shows a third end cross sectional view of the implant embodiment depicted in FIG. 5 A-FIG. 5D;

FIG. 7 G shows a fourth end cross sectional view of the implant embodiment depicted in FIG. 5 A-FIG. 5D;

FIG. 8 shows 4 alternate views of an assembly of the first component of the implant according to the disclosure and as shown in FIG. 6, the upper panel shows a first side view of an inserter instrument assembled with the first component, the left middle panel shows the assembly from a top (proximal end of the inserter instrument), the right middle panel shows the assembly from a bottom (distal end of the inserter instrument), and the bottom panel shows an exploded view of the first component on the left and the distal end of the inserter on the right;

FIG. 9 shows a top perspective view of the second component and a side view of the first component of the implant embodiment depicted in FIG. 5 A-FIG. 5D;

FIG. 10 shows a side perspective view of the second component and a side view of the first component of the implant embodiment depicted in FIG. 5 A-FIG. 5D, depicting rotation of the second component for insertion into the first component;

FIG. 11 shows a partial assembly of the implant embodiment depicted in FIG. 5 A-FIG. 5D, showing an end perspective view of the second component partially inserted in the first component (shown in side view);

FIG. 12 shows a partial assembly of the implant embodiment depicted in FIG. 5 A-FIG. 5D, showing side view of each of the second component and the first component after further rotation of the first component 90 degrees in each of its length and width dimensions;

FIG. 13 shows a bottom perspective view of the assembled first and second components shown in FIG. 9-FIG. 12;

FIG. 14 shows a side perspective view of the assembled first and second components shown in FIG. 9-FIG. 12 and the third component shown in FIG. 5 and FIG. 6 bottom panel, partially inserted in the first component;

FIG. 15 shows a side perspective view of the assembled first and second components shown in FIG. 9-FIG. 12 and the third component shown in FIG. 5 and FIG. 6 bottom panel, fully inserted in the first component; and

FIG. 16 shows an alternate side perspective view of the assembled first, second and third components of the implant embodiment depicted in FIG. 5 A-FIG. 5D and FIG. 15.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DESCRIPTION

In accordance with the various embodiments, techniques, instruments and implants are provided which can be used individually or together to facilitate the preparation of a spinal disc space for receiving an implant and enhancing the achievement of correction of sagittal balance within the spine. In some embodiments, the techniques enable enhanced preparation of the disc space. In other embodiments, the instruments provide features that are improved over the art and facilitate enhanced disc space preparation. And in yet other embodiments, the implants are adapted for implantation, particularly, though not exclusively through minimally invasive approaches and in situ assembly.

Technique and Instruments for Disc Preparation

In some embodiments, provided here is a technique that may be used, particularly during a TLIF procedure, for preparing the disc space for placement of an implant so as to optimize distraction of the disc space and disruption of the contralateral (in some cases, anterior) annulus. According to the technique, a conventional Collis-type Interbody retractor/distractor (essentially an elongate handle at its proximal end and having at its distal end a wide blade with a generally planar surface) is selected, whereby one or more such distractors are passed into the disc space and advanced toward the anterior annulus under X-ray (fluoroscopy) then rotated by 90 degrees to break calcifications and release the annulus. According to the technique, the interspace may be opened to provide enhanced distraction for implanting interbody devices to recreate the anatomically relevant lordosis.

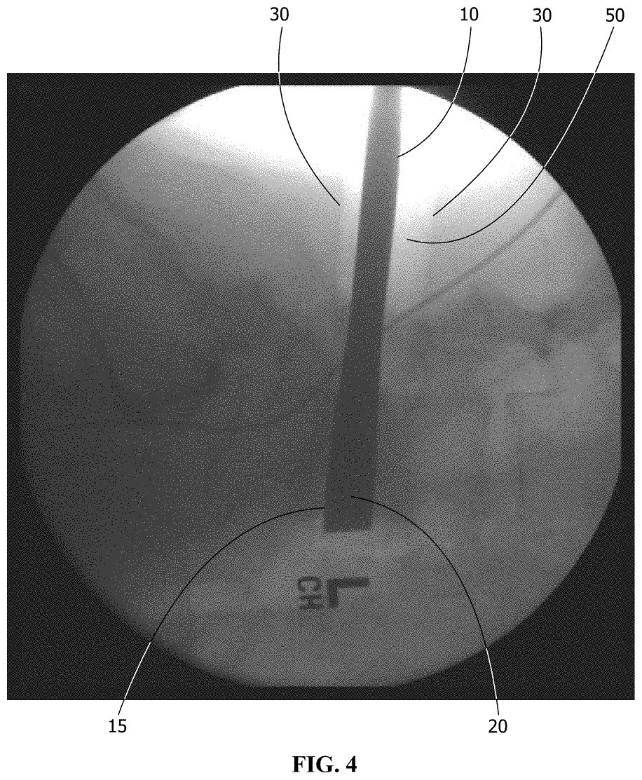

Referring now to the drawings, FIG. 4 shows a sagittal view (lateral) x-ray image showing a planar bladed distractor 10 as inserted in the disc space and rotated so that the planar surface 20 of its distal blade 15 is perpendicular to the vertebral endplates 30. In use according to the technique, at least a first planar bladed distractor 10 is inserted into the disc space 50 with the planar blade surface 20 initially parallel to the endplates 30 of the disc space 50, and passed through the disc space 50 until the blade is positioned just posterior to the anterior edge 60 of the vertebral body endplates 30 and adjacent to the anterior annulus. The planar bladed distractor 10 is then rotated 90 degrees such that the planar blade surface 20 is perpendicular to the endplates 30 of the vertebra to separate the vertebra and effectively “tear” the anterior annulus. In some embodiments, the technique involves sequential insertion of distractors with increasingly larger blades, each inserted side by side along an arc across the anterior annulus, to persuade distraction and disruption of the annulus. Position is confirmed under fluoroscopy.

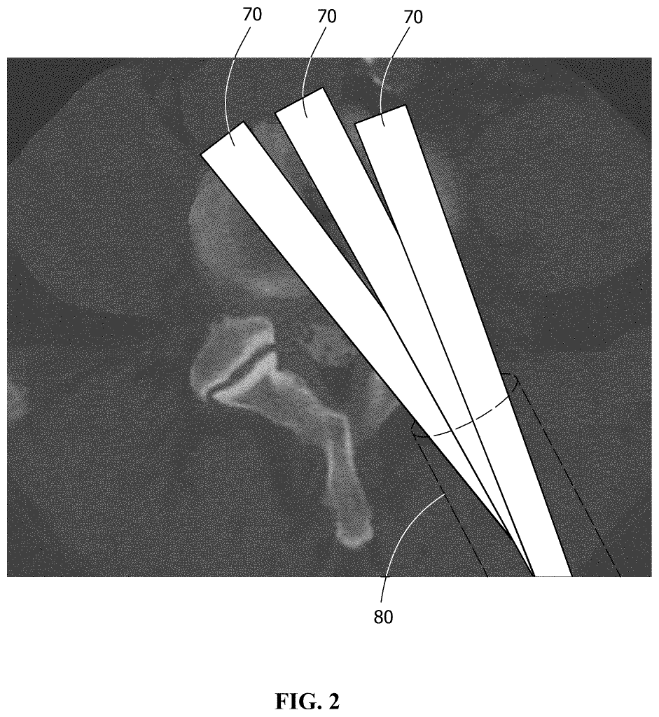

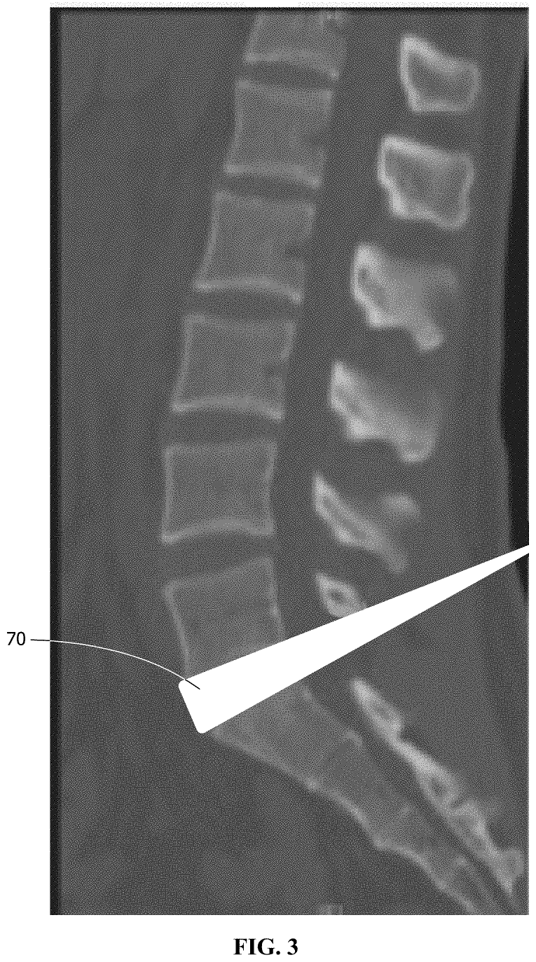

Referring now to FIG. 3 and FIG. 2, alternate schematic images are show with a generally wedge-shaped model 70 of a distractor shown, where FIG. 3 shows a single distractor positioned relative to the L4-L5 disc space as overlaid on a sagittal view radiographic image, and FIG. 2 shows an array of distractors positioned relative to the L4-L5 disc space as overlaid on an axial (AP) view radiographic image.

As schematically depicted in FIG. 2, in use, one or an array of distractors, such as planar bladed distractors 10, can be inserted and rotated across the disc space and within the constraints of a tubular retractor 80 (show in dotted lines) to achieve disruption of the annulus. Thus, it will be appreciated that an array of distractors may be used in parallel or series, or a single distractor may be used to disrupt the annulus across the disc space. The axial view shown in FIG. 2 illustrates an array of unrotated distractors, their planar blades depicted as wedges in axial view, traversing the disc space to the contra-lateral anterior annulus, and arrayed from the middle toward the ipsilateral side of the anterior annulus. In such embodiments where the procedure is done via a TLIF retractor tube, manipulation across the disc space is achieved by angling the side to side movement of the distal ends of each bladed retractor, within the limits of the tube or retractor. Referring again to FIG. 3, the schematic depicts a distractor after rotation within the disc space, its planar blade depicted as a wedge in sagittal view and FIG. 4 depicts positioning of the distraction for disruption of the anterior annulus at L4-L5. The technique can be used at any level and is particularly optimized for safety and effectiveness at L5-S1.

As is further described herein below, a disclosed embodiment of an articulating paddle distractor according to the disclosure enables a similar type of motion to disrupt the annulus using an articulating embodiment of the distractor to further enhance penetration into the disc space along the anterior annulus. Use of this alternate distractor is particularly advantageous in that it avoids the need for rotation of a blade against the vertebral endplates, which can cause undesirable damage to the endplate surface and possibly induce fracture. Further description of the paddle distractor follows.

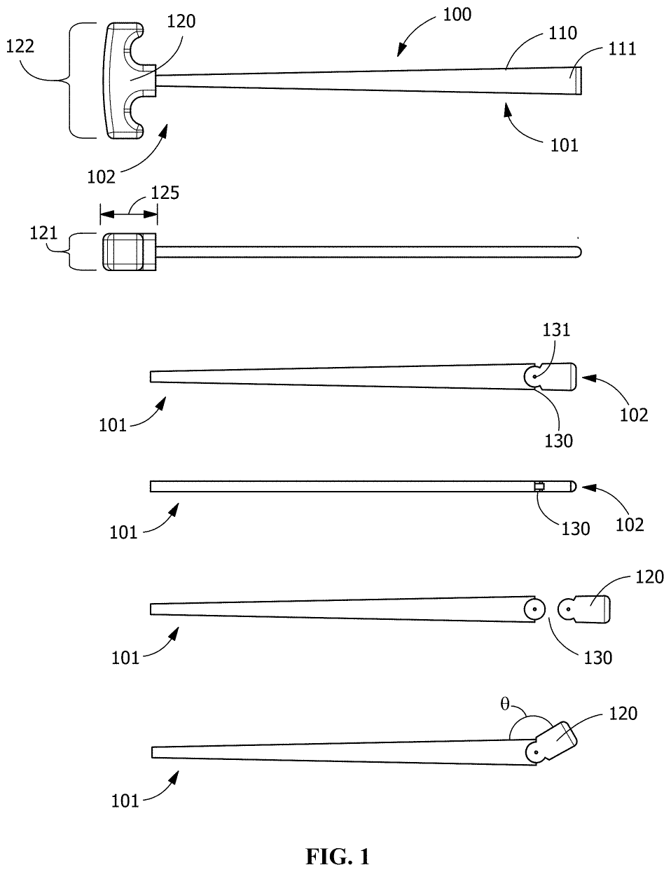

Referring again to the drawings, as shown in FIG. 1, an embodiment of a distractor 100 instrument according to the disclosure is provided. The instrument is adapted for insertion into the disc space to facilitate distraction and penetration or rupture of the annulus. The instrument may be used alone or as a set of two or more. As shown, the paddle distractor 100 is an instrument having an elongate handle 110 with a grip 111, which can include a cylindrical, knobbed, T-type or other type grip that is known in the art, at its proximal end 101, and at the tip its distal end 102 a flared paddle 120.

In the various embodiments, the flared paddle 120 has a width dimension in a first plane 121 that is generally the same as or slightly greater than the width dimension of the elongate handle 110 and/or the grip 111, and a length dimension in a second plane 122 that is oriented 90 degrees to the first plane that is greater than the width dimension in the first plane 121. The flared paddle 120 also has a height dimension 125 as measured from the distal most end of the flared base to its point of attachment 130 to the handle. In some embodiments, the height dimension 125 is less than or equal to the width dimension 124, and in other embodiments, the height dimension 125 is greater than the width dimension 124.

In some embodiments, the flared paddle 120 may be generally block shaped where its width in one or both of the first and second planes does not vary along the height dimension, such as is shown in the depicted embodiments of FIG. 1. In other embodiments, the flared paddle 120 may be overall wedge or trapezoidal shaped, tapering from wider to narrower in one or both of the width and length dimensions from distal to proximal.

Referring again to FIG. 1, as shown in the lower panel, in some embodiments the flared paddle 120 is adapted with a pivot joint 131 at an attachment point 130 with the elongate handle 110 to enable rotation of the flared paddle 120 from side to side from about 30 degrees of motion from the vertical (angle depicted in the drawings as θ). In some embodiments, the range of motion may be greater than 30 degrees and up to 90 degrees or it may be less than 30 degrees, such as 10, 15, 20 or 25 degrees.

Implants

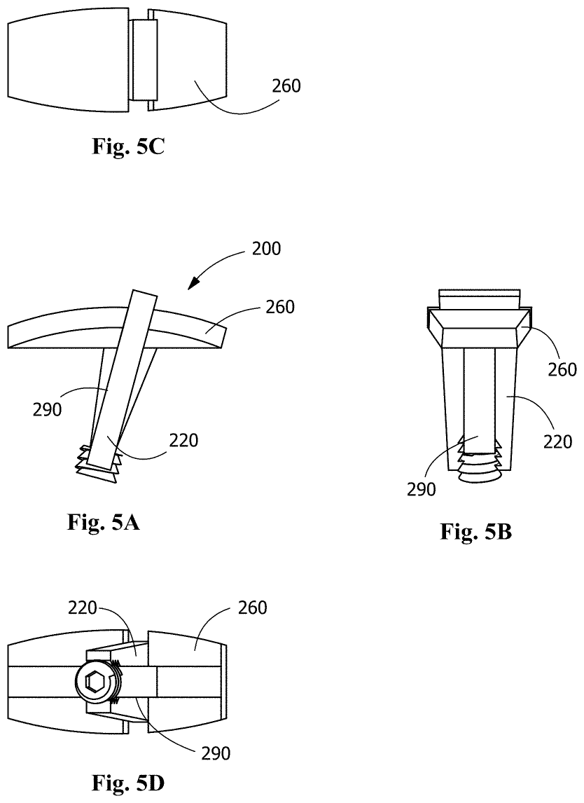

Provided are implants that can be assembled in situ, particularly in the context of a minimally invasive procedure, such as TLIF. Referring to FIG. 5, The implant includes a transverse component 260, a sagittal component 220, and in some embodiments, a locking component 290. When the modular implant is assembled the transverse component 260 is oriented orthogonally to the sagittal component 220. In some particular embodiments, as depicted in the drawings, the implants include three components, including a generally wedge shaped sagittal component 220, a generally transverse component 260 (when engaged, it is oriented transverse to the sagittal component), and a generally cylindrical locking component 290. Referring again to the drawings, FIG. 5 depicts, in four alternate views, an exemplary implant assembly 200 according to the disclosure. FIG. 5 A is a side view and depicts the implant assembly 200 as it would be positioned in a disc space as viewed along the axis of the spine (AP view), the figure showing the transverse component 260 at the top and the sagittal component extending downwardly. The upper left panel shows a side view clearly depicting the lordotic form of the sagittal component. The lower left panel depicts a view from an anterior perspective (across the disc space), and the lower right panel depicts a view from the posterior perspective.

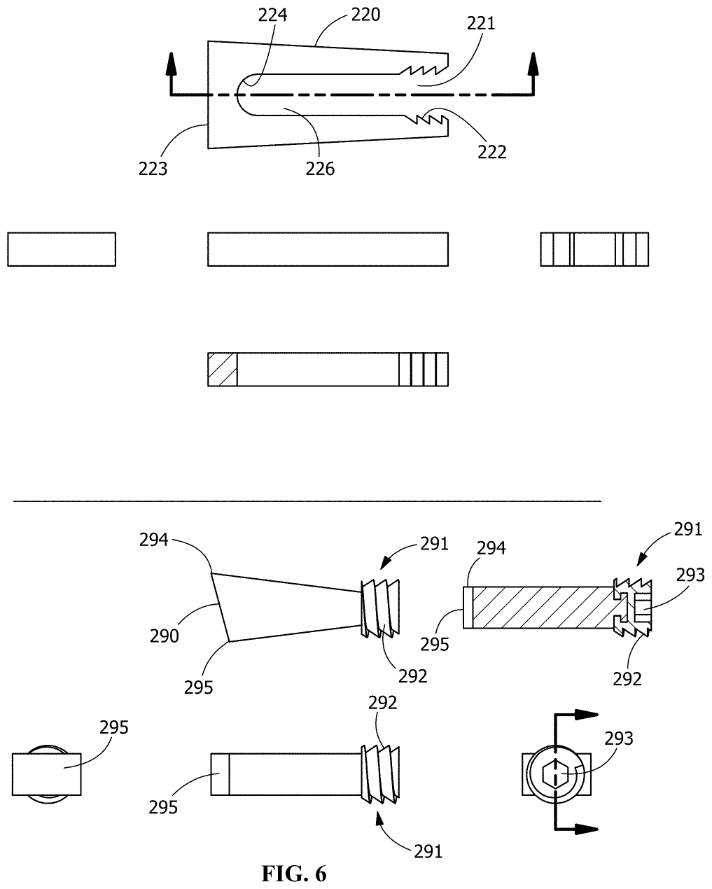

Referring now to FIG. 6, the sagittal component 220 is shown in alternate views in the upper panel of the figure, including side view (upper image), top view (center image), alternate end views (center left and center right, respectively the anterior- and posterior-oriented ends relative to a disc space), and cross-sectional view (lower image) sectioned along the lines shown in the upper image. As shown, the sagittal component 220 includes at its first, open end 221 an engagement feature 222 for fixation with the locking component 290, and a center slot 226. As depicted, the engagement feature 222 includes threads; however, in alternate embodiments, the engagement feature 222 may be selected from other suitable feature for fixed engagement with a complimentary feature on the locking component 290. The sagittal component 220 also includes at its closed end 223 a shaped receiving seat 224 for engagement with the transverse component 260. As depicted, the shaped receiving seat 224 has a generally U shape. It will be appreciated that in other embodiments, the shaped receiving seat 224 may be other than curved, and may be squared, or conical. As shown in the various drawings, the depicted embodiment of the sagittal component 220 has an overall lordotic shape wherein it tapers from broader at the closed end 223 to narrower at the open end 221. In alternate embodiments, the sagittal component 220 does not taper, or it may taper from a broader open end 221 to a narrower closed end 223.

Referring again to FIG. 6, the lower panel shows various views of the locking component 290, which has at its proximal end 291 an engagement portion 292, which is shown as threads, for engagement with the sagittal component 220. According to the embodiments shown in the drawings, the locking component 290 engagement portion 292 is threaded, and includes at an end feature 293 a tool engagement feature for actuating its engagement with the sagittal component 220. The locking component 290 includes at its distal end 294 a biasing edge 295 which operates to compress against the transverse component 260 when it is seated in the sagittal component 220, whereby locking engagement of the locking component 290 with the sagittal component 220 serves to lock and fix the transverse component 260 within the assembly 200. Of course, in alternate embodiments wherein the locking component 290 includes an engagement portion 292 that is not threaded, the end feature 293 may have a different configuration that may be engageable with a tool for actuation of engagement with the sagittal component 220.

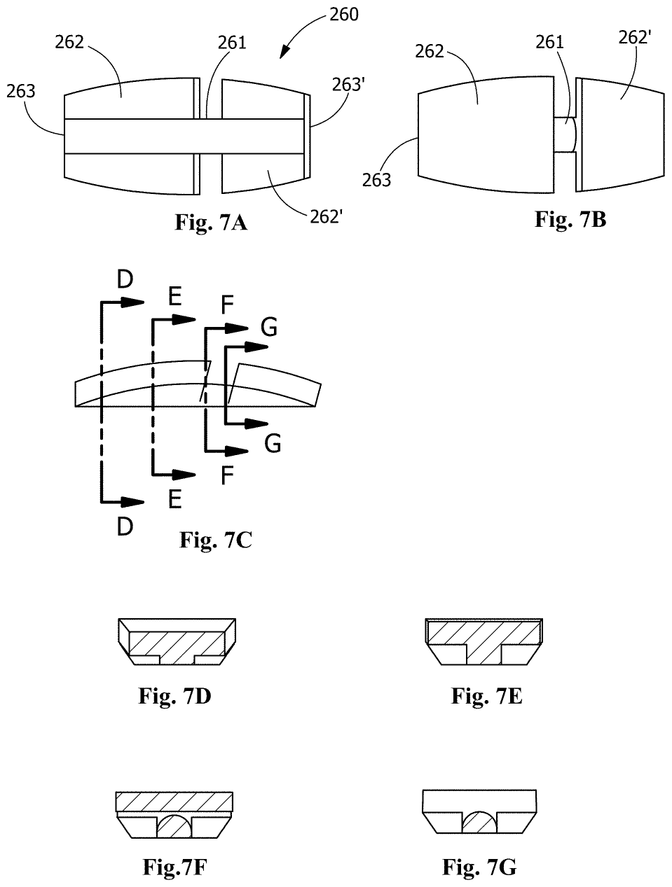

Referring now to FIG. 7, alternate top, posterior and anterior views of the transverse component 260 are shown. The component has a generally dumbbell shape with an emplacement 261 (depicted as a post or cylindrical rod-shaped portion) joining opposite generally planer members 262, 262′ that taper from the emplacement 261 to their ends 263, 263. As shown, each of the planar members 262, 262′ is generally trapezoidal shaped, and differ in their overall dimensions, where one is slightly larger than the other. According to the depicted embodiment of the transverse component 260, the width of the planer members 262, 262′ is greater than their thickness.

Of course, in alternate embodiments, the shapes of each member may be different from one another and their relative dimensions including width and thickness may vary from those as shown in the drawings. Moreover, though the transverse component is depicted as having a curved upper surface and a curved lower surface, and an overall arcuate shape, it will be appreciated that the shape may be flat and planar or may be flat with a convex upper surface. Further, while the emplacement is defined by apertures that extend towards the middle of the component from its sides (lateral edges) and are generally slot shaped, it will be appreciated that the apertures may be other than slot shaped. Further still, the emplacement may be offset from the center of the length dimension, as depicted in the embodiment shown in the drawings, or may be centered therein. And the emplacement may have a cross section that is generally circular (as depicted in the drawings), or square, rectangular, triangular, and the like.

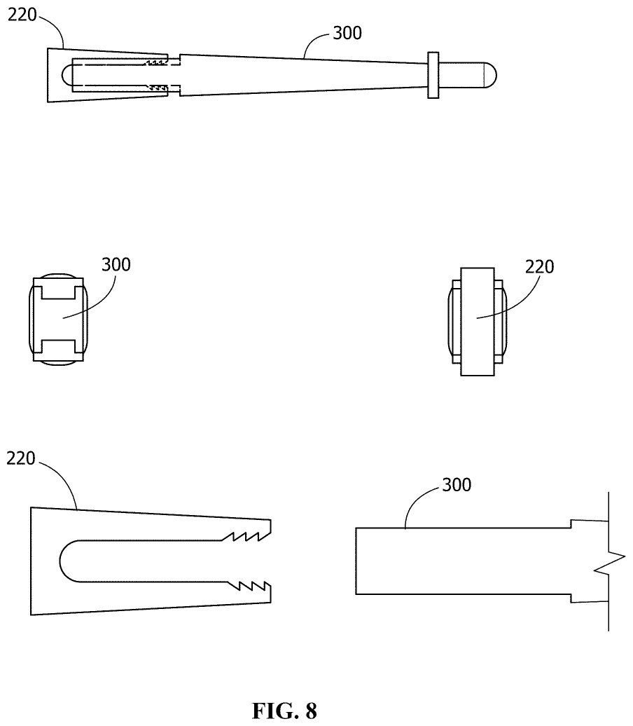

Referring again to the drawings, FIG. 8 shows in alternate views the sagittal component 220 as engageable with an elongate inserter tool 300. As depicted, FIG. 8 includes in the upper panel a first side view of an inserter instrument assembled with the sagittal component 220. In the left middle panel FIG. 8 shows the assembly of the inserter tool 300 and the sagittal component 220 from a top (proximal end of the inserter instrument), the right middle panel shows the assembly of the inserter tool 300 and the sagittal component 220 from a bottom (distal end of the inserter instrument), and the bottom panel shows the assembly of the inserter tool 300 and the sagittal component 220 in an exploded view, where the sagittal component 220 is on the left and the distal end of the inserter tool 300 is on the right.

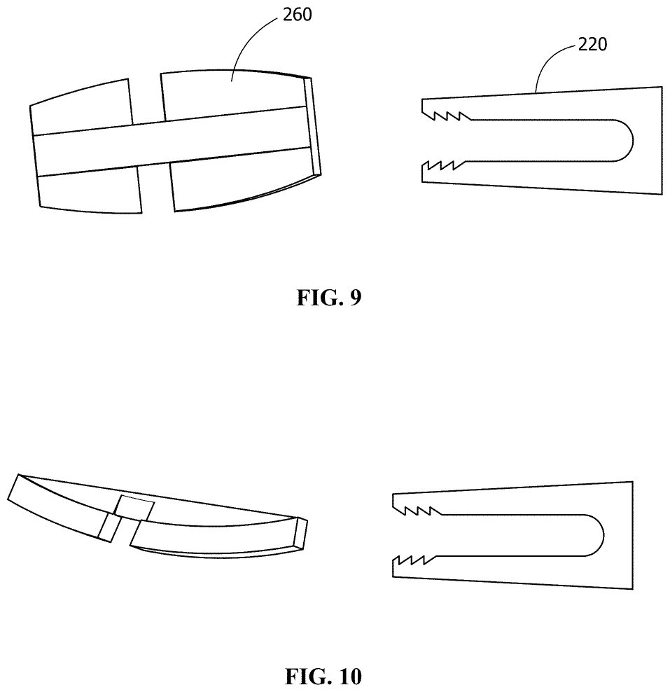

In an exemplary embodiment, as is generally depicted sequentially in FIG. 9-FIG. 12, the modular implant 200 includes a transverse component 260 having a length dimension, a width dimension and a depth dimension, wherein the length dimension is greater than the width dimension which is greater than the depth dimension, and having two planar members defined by apertures adjacent to the emplacement. The modular implant further includes a sagittal component 220 that includes a wedge member having a slot 226 that opens at the tip portion 221 and extends to the coupling surface 224 to define a first prong and a second prong on either side of the slot 226 and extending away from the coupling surface 224, wherein the slot 226 has a width dimension between the prongs that is larger than the depth dimension of the transverse component 260 and is smaller than the width dimension of the transverse component 260. According to such embodiment, the transverse component 260 is adapted for insertion into the slot 226 of the sagittal component 220 lengthwise with its length dimension parallel with the prongs, and then rotated by about 90 degrees so that its length dimension is perpendicular to the prongs and its width dimension is parallel with the prongs, and then rotated again by about 90 degrees so that its depth dimension is parallel with the prongs such that the transverse component 260 is oriented generally orthogonally with respect to the sagittal component 220.

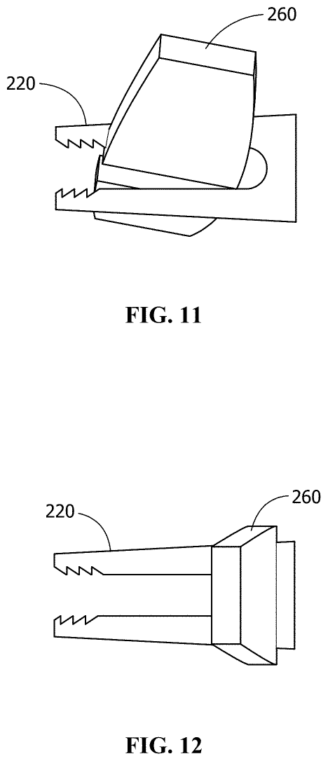

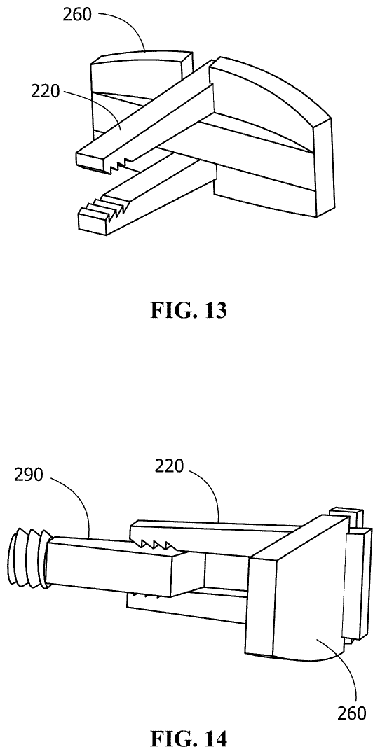

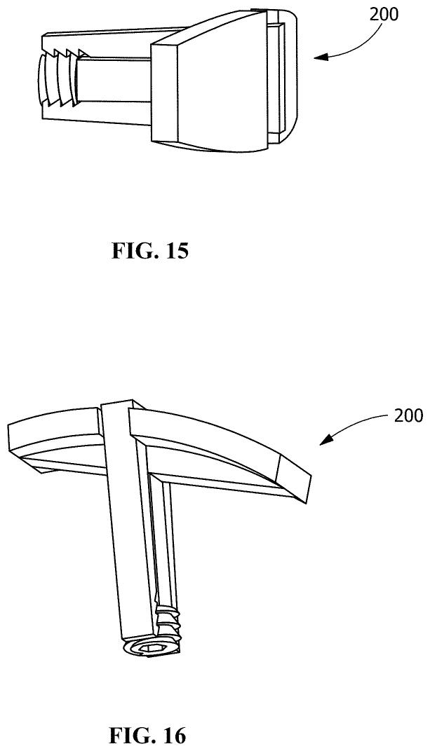

Thus, the implant 200 is designed so that it may be inserted piece by piece and assembled within a distracted disc space by first inserting the sagittal component 220 into the space with the elongate tool, the wider flat portion of the sagittal component 220 insertable in a first orientation into the disc space with its planar surfaces parallel to the vertebral endplates and directed toward the anterior wall where is it rotated 90 degrees by actuation of the inserter tool. Referring now to FIG. 9, the transverse component 260 is then introduced into the disc space with its planar surfaces parallel to the vertebral endplates and slid into the opening in the sagittal component 220 as shown in FIG. 10. The transverse component 260 is then rotated in the same plane by 90 degrees within the slot 226, as shown in FIG. 11, to orient the emplacement 261 adjacent the shaped receiving seat 224 in the sagittal component 220, and then is rotated again by 90 degrees, this time around its long axis such that the emplacement 261 is positioned in the shaped receiving seat 224 in the sagittal component 220 center slot 226, as shown in FIG. 12. The locking component 290 is then inserted into the center slot 226 of the sagittal component 220 as shown in FIG. 14 and is threaded into engagement with the sagittal component 220, as shown in FIG. 15. The assembled implant is shown in FIG. 16.

Of course, it will be appreciated that the implant assembly 200 may be assembled outside the disc space and subsequently inserted therein. Further, while the above description contemplates that that implant assembly 200 is positioned in the disc space with the transverse component 260 oriented toward the anterior aspect of the disc space, in alternate embodiments, the implant assembly 200 may be positioned in alternate orientations.

Embodiments of the present invention are not limited to use in a posterior or transforaminal approach for spinal surgery and may be adapted for uses in other spinal surgical orientations by other means of access within a disc space.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The general inventive concepts may be embodied in different forms and should not be construed as limited to the embodiments set forth herein. Rather, these embodiments are provided so that this disclosure will be thorough and complete, and will fully convey the scope of the general inventive concepts to those skilled in the art.

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point). The term “operator” means and refers to any professional or paraprofessional who delivers clinical care to a medical patient, particularly in connection with the delivery of care.

Anatomical references as used herein are intended to have the standard meaning for such terms as understood in the medical community. For example, the application may include reference to the following terms: “cephalad,” “cranial” and “superior” indicate a direction toward the head, and the terms “caudad” and “inferior” indicate a direction toward the feet. Likewise, the terms “dorsal” and “posterior” indicate a direction toward the back, and the terms “ventral” and “anterior” indicate a direction toward the front. And the term “lateral” indicates a direction toward a side of the patient. The term “medial” indicates a direction toward the mid line of the patient, and away from the side, the term “ipsalateral” indicates a direction toward a side that is proximal to the operator or the object being referenced, and the term “contralateral” indicates a direction toward a side that is distal to the operator or the object being referenced.

More specifically with respect to the directional movement of an implant according to the methods of the disclosure, sideways refers to the general direction of movement within the disc space between the endplates from the position of the inserted instruments toward one or the other of the contralateral and ipsilateral portions of the disc space. In the case of a TLIF procedure, such sideways motion will generally be in a medial direction relative to the disc space. Though in other types of surgical access, particularly within the spine, sideways movement may be either medial or lateral relative to the disc space, and in other surgical contexts sideways is away from the initial position of the implant. Further, with respect to the movement of an implant by action of the surgical instruments, the movement may also be rotational, wherein the action of the instruments directs the implant sideways and also in a rotational or pivotal motion. More generally, any and all terms providing spatial references to anatomical features shall have meaning that is customary in the art.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as may be described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach. Further, all references herein are made in the context of the representative images shown in the drawings. Fewer or additional instruments, including generic instruments, may be used according to the preference of the operator. Moreover, references herein to specific instruments are not intended to be limiting in terms of the options for use of other instruments where generic options are available, or according to the preference of the operator.

While the disclosed embodiments have been described and depicted in the drawings in the context of the human spine, it should be understood by one of ordinary skill that all or various aspects of the embodiments hereof may be used in in connection with other species and within any species on other parts of the body where deep access within the tissue is desirable.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

What is claimed is:

1. A modular spinal implant, comprising:
a transverse component having a generally convex top surface, a bottom surface, and an emplacement; and
a sagittal component having a generally wedge-shaped conformation defined by elongate sides and a wide base that tapers toward a tip portion, and a coupling surface at the base,
wherein the coupling surface of the sagittal component and the emplacement of the transverse component are arranged and disposed to engaged to form an attached configuration between the transverse component and the sagittal component, with the tip portion of the sagittal component extending from the bottom surface of the transverse component.

2. The modular spinal implant of claim 1, wherein the sagittal component includes a wedge member and a locking member, the locking member attaching to the wedge member in a locked configuration to retain the transverse component and the sagittal component in the attached configuration.

3. The modular spinal implant of claim 2, wherein either: one of the wedge member and the locking member includes a gear rack and the other of the wedge member and the locking member includes a ratchet; or the wedge member includes an internal threaded surface and the locking member includes an external threaded surface.

4. The modular spinal implant of claim 2, wherein the wedge member includes a slot that opens at the tip portion and extends to the coupling surface disposed, and a first prong and a second prong on either side of the slot and extending away from the coupling surface.

5. The modular spinal implant of claim 4, wherein the slot that extends to the coupling surface is generally U shaped.

6. The modular spinal implant of claim 4, wherein the locking member is arranged and disposed to occupy and substantially fill the slot in the locked configuration.

7. The modular spinal implant of claim 4, wherein the transverse component includes a first aperture and a second aperture adjacent to the emplacement, the first aperture being arranged and disposed to receive the first prong and the second aperture being arranged and disposed to receive the second prong in the attached configuration such that the coupling surface of the sagittal component contacts the emplacement of the transverse component.

8. The modular spinal implant of claim 7, wherein the first aperture and the second aperture extend from the emplacement to a lateral surface of the transverse component.

9. The modular spinal implant of claim 7, wherein the generally convex top surface of the transverse component includes a recess arranged and disposed to engage the base of the slot in the attached configuration.

10. The modular spinal implant of claim 1, wherein the sagittal component and the transverse component are arranged and disposed such that in the attached configuration a center line of the sagittal component extending from the bottom surface of the transverse component to the tip portion of the sagittal component is oblique relative to the bottom surface.

11. The modular spinal implant of claim 1, wherein the coupling surface of the sagittal component is on the base and includes an attachment feature that is complimentary with an attachment feature on the bottom surface of the transverse

12. A modular spinal implant of claim 1, wherein the transverse component has a length dimension, a width dimension and a depth dimension, and wherein the length dimension is greater than the width dimension which is greater than the depth dimension.

13. A modular spinal implant of claim 12, wherein the transverse component includes a first aperture and a second aperture adjacent to the emplacement to define two planar members, wherein the apertures and the emplacement are either centered or off center along the length dimension.

14. A modular spinal implant of claim 12, wherein the transverse component has a generally arcuate shape.

15. A modular spinal implant of claim 1, the transverse component having a length dimension, a width dimension and a depth dimension, wherein the length dimension is greater than the width dimension which is greater than the depth dimension, and having two planar members defined by apertures adjacent to the emplacement, and sagittal component including a wedge member having a slot that opens at the tip portion and extends to the coupling surface to define a first prong and a second prong on either side of the slot and extending away from the coupling surface, wherein the slot has a width dimension between the prongs that is larger than the depth dimension of the transverse component and is smaller than the width dimension of the transverse component 260, wherein, the transverse component is adapted for insertion into the slot of the sagittal component lengthwise with its length dimension parallel with the prongs, and then rotated by about 90 degrees so that its length dimension is perpendicular to the prongs and its width dimension is parallel with the prongs, and then rotated again by about 90 degrees so that its depth dimension is parallel with the prongs such that the transverse component is oriented generally orthogonally with respect to the sagittal component.

16. A method for surgically modifying a spine with a modular spinal implant, comprising:
inserting a transverse component into a distracted disc space of a spinal column, the transverse component having a generally convex top surface, a bottom surface, and an emplacement;
inserting a sagittal component into the distracted disc space, the sagittal component having a generally wedge-shaped conformation defined by elongate sides and a wide base that tapers toward a tip portion, and a coupling surface; and
attaching the sagittal component to the transverse component in the distracted disc space to form the modular spinal implant in an attached configuration with the coupling surface of the sagittal component engaged to the emplacement of the transverse component, and with the sagittal component extending from the bottom surface of the transverse component with the tip portion of the sagittal component distal from the bottom surface of the transverse component.

17. The method of claim 16, wherein the sagittal component includes wedge member and a locking member, and attaching the sagittal component to the transverse component includes attaching the locking member to the wedge member to form a locked configuration retaining the transverse component and the sagittal component in the attached configuration.

18. The method of claim 17, wherein the wedge member is inserted into the distracted disc space prior to the transverse component being inserted into the distracted disc space.

19. The method of claim 18, wherein the locking member is inserted into the distracted disc space following the transverse component being inserted into the distracted disc space.

20. The method of claim 19, wherein:
the transverse component includes a first aperture and a second aperture adjacent to the emplacement;
the wedge member includes a slot that opens at the tip portion and extends to a base of the slot, and a first prong and a second prong on either side of the slot; and
attaching the sagittal component to the transverse component includes inserting the first prong into the first aperture and the second prong into the second aperture such that the coupling surface of the sagittal component contacts the emplacement of the transverse component.

21. The method of claim 20, wherein the first aperture and the second aperture extend from the emplacement to a lateral surface of the transverse component, and inserting the first prong into the first aperture and the second prong into the second aperture includes rotating the transverse component relative to the wedge member and then inserting the locking member into the slot.

22. The method of claim 21 wherein inserting the locking member into the slot occupies and substantially fills the slot.

23. The method of claim 21, wherein inserting the first prong into the first aperture and the second prong into the second aperture includes engaging the base of the slot with a recess disposed in the generally convex top surface of the transverse component.

24. The method of claim 16, wherein either: one of the wedge member and the locking member includes a gear rack and the other of the wedge member and the locking member includes a ratchet, and attaching the locking member to the wedge member includes engaging the ratchet and the gear rack; or the wedge member includes an internal threaded surface and the locking member includes an external threaded surface, and attaching the locking member to the wedge member includes engaging the external threaded surface with the internal threaded surface.

25. A method for surgically preparing a spinal disc space, comprising the steps:
(i) directing at least a first elongate distractor blade having a proximal end and a distal end comprising a flared paddle into a spinal disc space and between adjacent vertebral endplates, the flared paddle of the distractor blade being parallel to the endplates;
(ii) advancing the flared paddle into contact with an anterior annulus of the spinal disc space;
(iii) actuating the distractor blade by rotation of its proximal end by about 90 degrees whereby the flared paddle is oriented rotated to be approximately perpendicular to the vertebral endplates;
(iv) optionally introducing at least a second elongate distractor blade adjacent to the first distractor blade, and repeating each of steps (i), (ii) and (iii), the at least second distractor blade having a flared paddle that is one of dimensionally larger, equivalent to or smaller than the flared paddle of the first distractor;
(v) optionally repeating step (vi) with one or more additional elongate distractor blades, each one or more additional elongate distractor blades having a flared paddle that one of dimensionally larger, equivalent to or smaller than the flared paddle of a previously inserted elongate distractor blade;
(vi) withdrawing each of the one or more of the elongate distractor blades;
(vii) optionally, repeating the steps (i)-(v), one or more times.
